(19) United States
(12) Patent Application Publication
(10) Pub. No.: US 2024/0382144 A1
(43) Pub. Date: Nov. 21, 2024

(54) WAVEFORM GENERATION IDENTIFYING METHOD AND COMPUTER-READABLE MEDIUM

(71) Applicants: Ryoji HIRANO, Tokyo (JP); Miyako ASAI, Osaka (JP); Masayuki HIRATA, Osaka (JP)

(72) Inventors: Ryoji HIRANO, Tokyo (JP); Miyako ASAI, Osaka (JP); Masayuki HIRATA, Osaka (JP)

(21) Appl. No.: 18/655,347

(22) Filed: May 6, 2024

(30) Foreign Application Priority Data
May 19, 2023 (JP) 2023-083209

(51) Int. Cl. A61B 5/00; A61B 5/245; A61B 5/372

(52) U.S. Cl. CPC A61B 5/4094; A61B 5/245; A61B 5/372; A61B 2562/0223

(57) ABSTRACT

A waveform generation identifying method includes: acquiring waveform data of a biosignal measured by a plurality of sensors; calculating distribution information indicating a distribution of values of the biosignal based on waveform data at a time point when characteristic waveform information of Interictal Epileptiform Discharge (IED) manifests, among the acquired waveform data; and giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.

### CROSS-REFERENCE TO RELATED APPLICATION

The present application claims priority under 35 U.S.C. § 119 to Japanese Patent Application No. 2023-083209, filed on May 19, 2023. The contents of which are incorporated herein by reference in their entirety.

### BACKGROUND OF THE INVENTION

### 1. Field of the Invention

The present invention relates to a waveform generation identifying method and a computer-readable medium.

### 2. Description of the Related Art

Clinical diagnosis of epilepsy using a magnetoencephalograph and an electroencephalograph includes evaluation of localization of an epileptic lesion in the brain using a technique referred to as an equivalent current dipole method. The equivalent current dipole method includes an estimation of a current source (dipole) that generates a magnetic field measured on the scalp. In order to estimate the dipole, it is necessary to identify, among time series data detected by a plurality of sensors, a time point when characteristic waveform information referred to as Interictal Epileptiform Discharge (IED) manifests, and a sensor that detects that the waveform information manifests. While analyzing characteristic waveforms in the analysis using a magnetoencephalograph and an electroencephalograph is very important, the sampling frequency and the number of sensors at the time of measurement tend to increase due to the progress of technology, leading to a tendency of an increased time need for visual search of the characteristic waveform information of IED. At present, these operations are performed manually by a medical doctor, in which magnetoencephalography data of enormous volume makes it difficult to manually and accurately identify the time point and the sensor regarding the characteristic waveform information of the individual IED.

As a technique of identifying the time point when and the sensor where a waveform characteristic of IED occurs, there is disclosed a technique of inferring an IED probability map using a trained model with machine learning and selecting a time point when and a sensor where characteristic waveform information of IED manifests (for example, Japanese Unexamined Patent Application Publication No. 2021-069929).

However, the technology described in Japanese Unexamined Patent Application Publication No. 2021-069929 performs selection of the sensor based on a machine learning model, having a problem that there is a case where the selection of the sensor is not necessarily concordant with a determination criterion of a medical doctor. This leads to a demand for providing an accurate method of sensor selection achieving a result close to medical doctor's determination criteria so as to facilitate medical doctor's intuitive determination.

### SUMMARY OF THE INVENTION

According to an aspect of the present invention, a waveform generation identifying method includes: acquiring waveform data of a biosignal measured by a plurality of sensors; calculating distribution information indicating a distribution of values of the biosignal based on waveform data at a time point when characteristic waveform information of Interictal Epileptiform Discharge (IED) manifests, among the acquired waveform data; and giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.

### BRIEF DESCRIPTION OF THE DRAWINGS

FIG. 1 is a diagram illustrating an example of an overall configuration of a biosignal measurement system according to a first embodiment;

FIG. 2 is a diagram illustrating an example of a configuration of functional blocks of a server according to the first embodiment;

FIG. 3 is a diagram illustrating an example of a hardware configuration of an information processing device according to the first embodiment;

FIG. 4 is a diagram illustrating an example of a configuration of functional blocks of the information processing device according to the first embodiment;

FIG. 5 is a diagram illustrating an example of a magnetic isofield map at a specific time point;

FIG. 6 is a diagram illustrating an example of a sensor selection region corresponding to a dipole pattern;

FIG. 7 is a flowchart illustrating an example of a flow of training processing in the information processing device according to the first embodiment;

FIG. 8 is a flowchart illustrating an example of a flow of dipole estimation processing in the information processing device according to the first embodiment;

FIG. 9 is a flowchart illustrating an example of a flow of dipole pattern detection processing in the information processing device according to the first embodiment;

FIG. 10 is a diagram illustrating an example of a configuration of functional blocks of a server according to a second embodiment;

FIG. 11 is a diagram illustrating an example of an onset search range in waveform data;

FIG. 12 is a flowchart illustrating an example of a flow of dipole estimation processing in the information processing device according to the second embodiment;

FIG. 13 is a flowchart illustrating an example of a flow of dipole pattern detection processing in an information processing device according to a third embodiment;

FIG. 14 is a diagram illustrating an example of a plurality of sensor selection regions corresponding to a dipole pattern; and

FIG. 15 is a flowchart illustrating an example of a flow of dipole estimation processing of an information processing device according to a fourth embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to provide a waveform generation identifying method and a computer-readable medium capable of accurately identifying a sensor where characteristic waveform information of IED manifests among a plurality of sensors used for waveform data measurement.

Hereinafter, embodiments of a waveform generation identifying method and a program according to the present invention will be described in detail with reference to the drawings. In addition, the present invention is not limited by the following embodiments, and constituent elements in the following embodiments include those that can be easily conceived by those skilled in the art, those that are substantially the same, and those within an equivalent range. Furthermore, various omissions, substitutions, alterations, and combinations of components can be made without departing from the scope of the following embodiments.

### First Embodiment

### Overall Configuration of Biosignal Measurement System

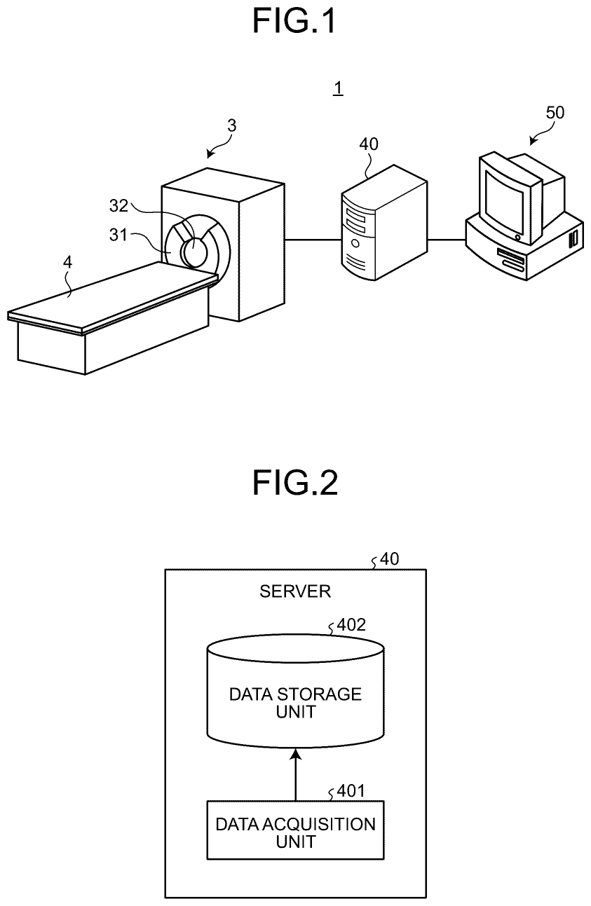

FIG. 1 is a diagram illustrating an example of an overall configuration of a biosignal measurement system according to a first embodiment. FIG. 2 is a diagram illustrating an example of a configuration of functional blocks of a server according to the first embodiment. An overall configuration of a biosignal measurement system 1 according to the present embodiment will be described with reference to FIGS. 1 and 2.

The biosignal measurement system 1 is a system that measures and displays a plurality of types of biosignals (for example, a magnetoencephalography (MEG) signal and an electroencephalography (EEG) signal) of a subject, emitted from a specific signal source (biological site). Note that the biosignal to be measured is not limited to the MEG signal or the EEG signal, and may be, for example, an electrical signal (electrical signal that can be expressed as an electrocardiogram) generated with the activity of the heart.

As illustrated in FIG. 1, the biosignal measurement system 1 includes: a measurement device 3 that measures one or more biosignals of the subject; a server 40 that records one or more types of biosignals measured by the measurement device 3; and an information processing device 50 which is a biosignal display device that analyzes the one or more types of biosignals recorded by the server 40. The measurement device 3 is a magnetoencephalograph that measures a MEG signal (an example of a biosignal) generated at the timing of applying a brain magnetic field or a stimulus, for example. Although FIG. 1 illustrates the server 40 and the information processing device 50 separately, it is also allowable to use a configuration in which at least a part of the functions of the server 40 is incorporated in the information processing device 50.

In the example of FIG. 1, the subject (measurement target person) lies on their back on a measurement table 4 with brain wave measurement electrodes (or sensors) attached to their head, and puts their head in a recess 32 of a Dewar 31 of the measurement device 3. The Dewar 31 is a retention container at a cryogenic temperature using liquid helium. Inside the recess 32 of the Dewar 31, a large number of magnetic sensors (for example, a Superconducting Quantum Interference Device (SQUID) sensor) for MEG measurement are arranged. The measurement device 3 collects the EEG signal from the electrode and the MEG signal from the magnetic sensor, and outputs time series waveform data including the collected EEG signal and MEG signal (hereinafter, simply referred to as waveform data in some cases) to the server 40. The waveform data output to the server 40 is read by the information processing device 50 to be displayed and analyzed. Typically, the Dewar 31 incorporating a magnetic sensor, and the measurement table 4, are disposed in a magnetic shield room, although the magnetic shield room is not illustrated in FIG. 1 for convenience.

The information processing device 50 is a device that analyzes waveform data of MEG signals from a plurality of magnetic sensors and waveform data of EEG signals from a plurality of electrodes. For example, the information processing device 50 synchronously displays the waveform data of MEG signals and the waveform data of EEG signals on a same time axis. Here, the EEG signal is a signal representing an electrical activity of a nerve cell (a flow of ionic charges generated in dendrites of neurons at the time of synaptic transmission) as a voltage value between electrodes. In addition, the MEG signal is a signal representing a minute electric field fluctuation caused by electrical activities of the brain.

As described above, the related art describes simultaneous implementation of selection of the time point when and sensor where characteristic waveform information of IED and by using a model trained with machine learning. Among such models, for example, a model such as a segmentation model is a simple model for selecting a time point and a sensor for dipole estimation, with low costs for training and inference. However, since it is applied in machine learning, there should be a possibility of outputting an undesirable inference result depending on training data used for the training. Usually, selection of a sensor is performed in a rectangular shape. However selection might be performed as a distorted selection region and a hollow selection region in some cases, causing a deviation from experience of a medical doctor performing a normal operation, leading to a possibility of lowering the reliability of the system. Above all, there is a possibility that a dipole estimation solution be obtained as an estimation at an inappropriate location affected by noise. In addition, when sensor selection has not been performed in many cases at the stage of using training data, output of the inference result is also likely to be performed without sensor selection. The guidelines of the American Clinical MEG Society (ACMEGS) (refer to Bagic, Anto I., et al. “American clinical magnetoencephalography society clinical practice guideline 1: recording and analysis of spontaneous cerebral activity.” Journal of Clinical Neurophysiology 28.4 (2011): 348-354) recommend sensor selection at dipole estimation for more accurate epileptic focus diagnosis. In addition, the guideline also describes the necessity of determining, at the time of dipole estimation, an appropriate time point in an abnormal signal, particularly between an onset zone and a peak zone of IED. Here, the onset zone indicates a starting portion of a waveform with a pointed peak, which is characteristic of IED, and the peak zone indicates a portion of the pointed peak of the waveform. The medical doctor calculates an appropriate time point between the onset zone and the peak zone based on the reliability of the current dipole, the amplitude of the magnetic field time series, and the like. In a case where the abnormal signal is nonstationary, it is necessary to select a sensor at each time point. It is possible to dynamically select a sensor at each time point even with the method in Japanese Unexamined Patent Application Publication No. 2021-069929 described above. However, there is a possibility of improving suitability of selection of a sensor by using a distribution of a magnetic field at each time point. The information processing device 50 according to the present embodiment is provided to improve the accuracy of sensor selection by using the distribution of the magnetic field at each time point.

As illustrated in FIG. 2, the server 40 includes a data acquisition unit 401 and a data storage unit 402.

The data acquisition unit 401 is a functional unit that periodically acquires waveform data of signals such as a MEG signal and an EEG signal measured by the measurement device 3. The waveform data includes time series data of each MEG signal measured by a plurality of magnetic sensors of the Dewar 31 of the measurement device 3 and time series data measured by a plurality of brain wave measurement electrodes attached to the head of the subject (measurement target person).

The data storage unit 402 is a functional unit that stores the waveform data acquired from the measurement device 3.

Although FIG. 1 illustrates a configuration in which the measurement device 3 and the server 40 are directly connected, and the server 40 and the information processing device 50 are directly connected, it is allowable to use a configuration in which data communication can be performed with each other via a network. Note that the network connection method may be a wired or wireless connection. Furthermore, the server 40 may also be a server on a network, and it is allowable to connect to the server 40 as a cloud network.

### Hardware Configuration of Information Processing Device

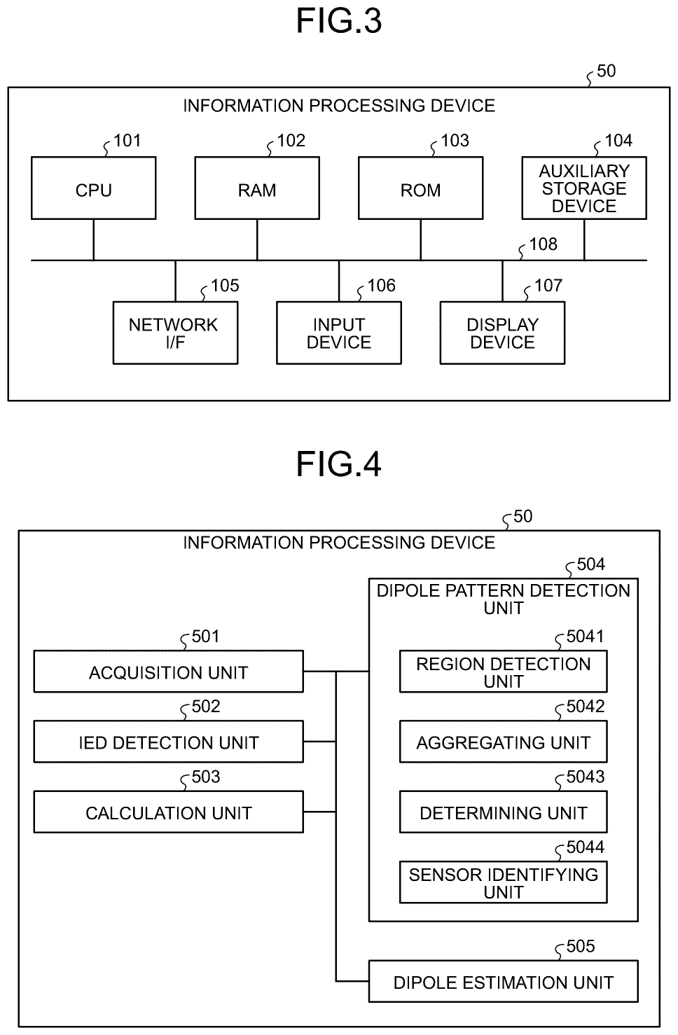

FIG. 3 is a diagram illustrating an example of a hardware configuration of an information processing device according to the first embodiment. A hardware configuration of the information processing device 50 according to the present embodiment will be described with reference to FIG. 3.

As illustrated in FIG. 3, the information processing device 50 includes a central processing unit (CPU) 101, random access memory (RAM) 102, read only memory (ROM) 103, an auxiliary storage device 104, a network I/F 105, an input device 106, and a display device 107.

The CPU 101 is an arithmetic device that controls the entire operation of the information processing device 50. The RAM 102 is a volatile storage device used as a work area of the CPU 101. The ROM 103 is a non-volatile storage device that stores a program to be used by the information processing device 50.

The auxiliary storage device 104 is a storage device such as a hard disk drive (HDD) or a solid state drive (SSD) that stores various data, programs, and the like.

The network I/F 105 is an interface for performing data communication with an external device such as the server 40 via a network. The network I/F 105 corresponds to the Ethernet (registered trademark), for example, and is implemented by a network interface card (NIC) or the like capable of wired communication or wireless communication conforming to transmission control protocol (TCP)/Internet protocol (IP) or the like.

The input device 106 is an input function of a touch panel, a mouse, a keyboard, or the like provided to select characters, numbers, various instructions, and move a cursor, or the like.

The display device 107 is a display constituted with liquid crystal, organic electro-luminescence (EL), or the like so as to display various types of information such as a cursor, a menu, a window, a character, or an image.

The CPU 101, the RAM 102, the ROM 103, the auxiliary storage device 104, the network I/F 105, the input device 106, and the display device 107 described above are communicably connected to each other by a bus 108 such as an address bus and a data bus.

The hardware configuration of the information processing device 50 illustrated in FIG. 3 is an example, and does not need to include all the components illustrated in FIG. 3, or may include other components.

### (Configuration and Operation of Functional Block of Information Processing Device)

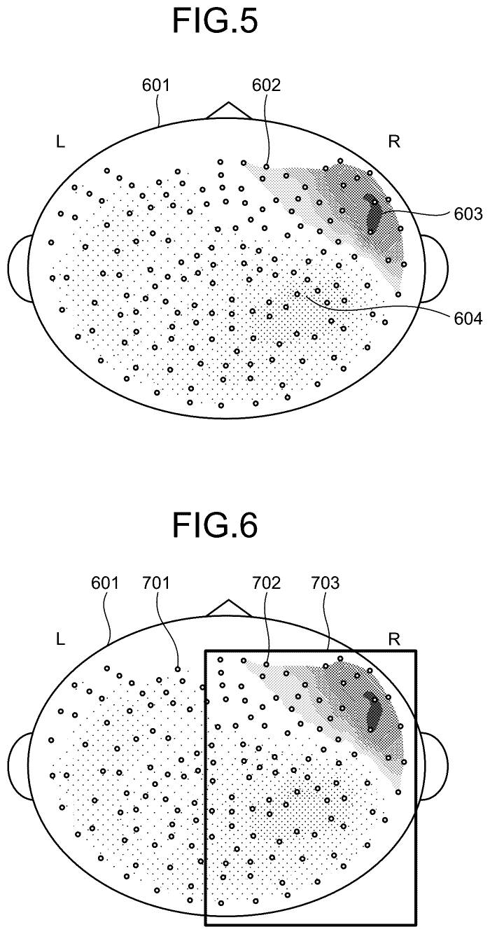

FIG. 4 is a diagram illustrating an example of a configuration of functional blocks of the information processing device according to the first embodiment. FIG. 5 is a diagram illustrating an example of a magnetic isofield map at a specific time point. FIG. 6 is a diagram illustrating an example of a sensor selection region corresponding to a dipole pattern. The configuration and operation of the functional blocks of the information processing device 50 according to the present embodiment will be described with reference to FIGS. 4 to 6.

As illustrated in FIG. 4, the information processing device 50 includes an acquisition unit 501, an IED detection unit 502, a calculation unit 503, a dipole pattern detection unit 504, and a dipole estimation unit 505.

The acquisition unit 501 is a functional unit that acquires waveform data of the MEG signal and the EEG signal from the server 40 via the network I/F 105.

The IED detection unit 502 is a functional unit that detects a time point when characteristic waveform information of IED manifests, referred to as a spike in the waveform data acquired by the acquisition unit 501. For the time point detection by the IED detection unit 502, for example, the method of Japanese Unexamined Patent Application Publication No. 2021-069929 described above can also be adopted.

The calculation unit 503 is a functional unit that calculates a magnetic isofield map (an example of distribution information) in which regions of equal magnetic field values are expressed by a same luminance value, based on the waveform data at the time point detected by the IED detection unit 502.

An example of the magnetic isofield map calculated by the calculation unit 503 is illustrated in FIG. 5. In this manner, the magnetic isofield map is usually calculated based on the magnetic field values measured by each of the plurality of sensors 602 of the magnetoencephalograph, the sensors 602 being disposed in planner arrangement as in the drawing of a human head 601. The magnetic isofield map as illustrated in FIG. 5 includes: a source 603, which is a region where the value of the magnetic field is larger than the surroundings; and a sink 604, which is a region where the value of the magnetic field is smaller than the surroundings. That is, in the magnetic isofield map at the time point when characteristic waveform information of IED manifests, a magnetic field pattern (hereinafter, may be referred to as a dipole pattern) including the source and sink of the magnetic field is observed.

The dipole pattern detection unit 504 is a functional unit that detects a dipole pattern region from the magnetic isofield map calculated by the calculation unit 503. Specifically, the dipole pattern detection unit 504 obtains, for example, a rectangular region (hereinafter, may be referred to as a sensor selection region) surrounding the sensor constituting the dipole pattern region from the magnetic isofield map calculated by the calculation unit 503, thereby identifying the sensor constituting the dipole pattern region. That is, the sensor included in the sensor selection region is the sensor to be identified by the dipole pattern detection unit 504. FIG. 6 illustrates an example of the sensor selection region obtained by the dipole pattern detection unit 504. In FIG. 6, the sensor selection region obtained by the dipole pattern detection unit 504 is illustrated as a sensor selection region 703. That is, a sensor 702 included in the sensor selection region 703 is the sensor identified by the dipole pattern detection unit 504, while the sensor not included in the sensor selection region 703 is the sensor not identified by the dipole pattern detection unit 504.

As illustrated in FIG. 4, the dipole pattern detection unit 504 includes a region detection unit 5041, an aggregating unit 5042, a determining unit 5043, and a sensor identifying unit 5044.

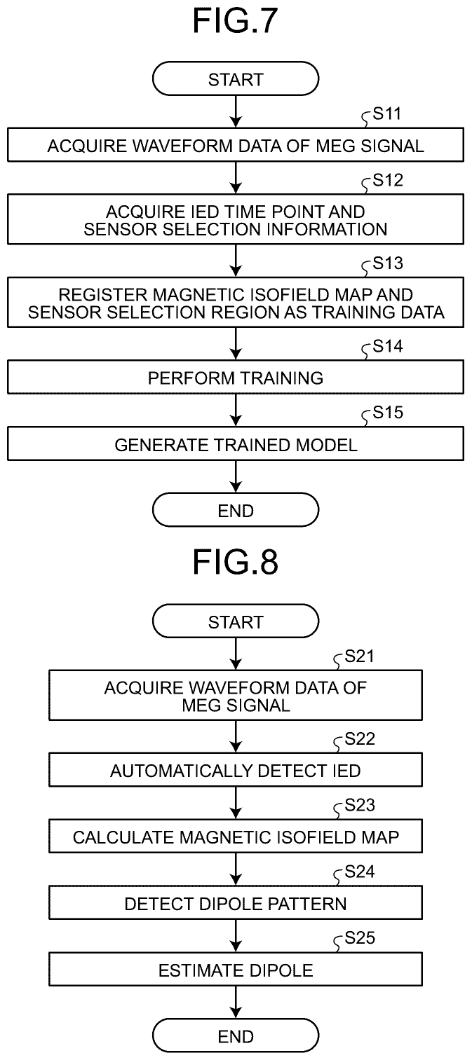

The region detection unit 5041 is a functional unit that gives the magnetic isofield map calculated by the calculation unit 503 as an input to a trained model to be described below, and detects a sensor selection region as an output from the trained model. Using waveform data that has undergone clinical dipole analysis of epilepsy by an expert such as a medical doctor, the trained model is trained to learn a magnetic isofield map of the waveform data and information of a sensor selected at the time of analysis, as training data (teaching data). When the sensor selection region exists, the sensor selection region may be used as the information regarding the selected sensor. When the sensor selection region does not exist, the sensor selection region may be restored from the selected sensor. That is, the model is trained using, as teaching data, data obtained by adding a sensor selection region as a label to a magnetic isofield map. The trained model generated by this training uses a magnetic isofield map an input, and obtains a sensor selection region as an output. Specific processing of the training of such a model will be described in detail with reference to FIG. 7.

Although the example illustrated in FIG. 5 described above includes the magnetic isofield map in which the arrangement of the three-dimensional sensors is put on a two-dimensional plane, it is also possible to use the arrangement of the three-dimensional sensors as it is in the training. In this case, it is possible to generate a trained model that similarly outputs a sensor selection region by using, as an input, a spatial magnetic isofield map represented in three dimensions labeled with a sensor selection region being a region of an ellipsoid sphere or a polyhedron including the selected sensor, in the training. However, the following description continues assuming that the magnetic isofield map is two-dimensional.

Furthermore, as a model to be trained, it is also possible to use a method with deep learning, such as: EfficientDet (refer to Tan, Mingxing, Ruoming Pang, and Quoc V. Le. “EfficientDet: Scalable and efficient object detection.” Proceedings of the IEEE/CVF conference on computer vision and pattern recognition. 2020) or YOLO (refer to Kim, Jun-Hwa, et al. “Object detection and classification based on YOLO-V5 with improved maritime dataset.” Journal of Marine Science and Engineering 10.3 (2022): 377), a method of training the model to learn a dipole pattern template, a method combining a method by machine learning, such as a Histogram of Oriented Gradients (HOG) features or a Support Vector Machine (SVM).

Although the above description uses the rectangular region as the sensor selection region, it is also possible to use an ellipse, a polygon, or the like as the sensor selection region.

In addition, some medical doctors perform dipole estimation using sensors included in a plurality of sensor selection regions. Even in this case, the plurality of sensor selection regions can be directly used for the training. Incidentally, a case of using a sensor selection region other than a single rectangle cannot be handled with an object detection model such as EfficientDet, and using Semantic Segmentation such as U-Net (refer to Ronneberger, Olaf, Philipp Fischer, and Thomas Brox. “U-net: Convolutional networks for biomedical image segmentation.” Medical Image Computing and Computer-Assisted Intervention-MICCAI 2015: 18th International Conference, Munich, Germany, Oct. 5-9, 2015, Proceedings, Part III 18. Springer International Publishing, 2015, for example), it is possible to perform operation similarly to object detection.

The aggregating unit 5042 is a functional unit that performs removal/aggregating of a plurality of sensor selection regions when the region detection unit 5041 has detected the plurality of sensor selection regions from one magnetic isofield map. Specifically, in a case where a plurality of sensor selection regions overlaps each other, the aggregating unit 5042 aggregates the sensor selection regions into one sensor selection region, and in a case where the detection probability is low in the detected sensor selection region, the aggregating unit removes the sensor selection region with low probability. The aggregating unit 5042 is only required to perform at least one of aggregating and removal. Furthermore, examples of an algorithm for performing removal/aggregating of the plurality of sensor selection regions include Non-Maximum Suppression (NMS), Non-Maximum Weighted (NMW), Weighted Boxes Fusion (WBF), and the like (refer to Solovyev, Roman, Weimin Wang, and Tatiana Gabruseva. “Weighted boxes fusion: Ensembling boxes from different object detection models.” Image and Vision Computing 107 (2021): 104117, for example).

The determining unit 5043 is a functional unit that determines whether the sensor selection region remains as a result of removal and aggregating of the sensor selection region by the aggregating unit 5042.

The sensor identifying unit 5044 is a functional unit that identifies a sensor included in the sensor selection region as a sensor constituting the dipole pattern region in a case where the determining unit 5043 has determined that the sensor selection region remains.

The dipole estimation unit 505 is a functional unit that performs dipole estimation using a time point when characteristic waveform information of IED manifests, identified by the IED detection unit 502 and waveform data corresponding to the sensor identified by the dipole pattern detection unit 504.

The acquisition unit 501, the IED detection unit 502, the calculation unit 503, the dipole pattern detection unit 504, and the dipole estimation unit 505 described above are implemented by executing a program by the CPU 101 illustrated in FIG. 3. Some or all of the functional units of the acquisition unit 501, the IED detection unit 502, the calculation unit 503, the dipole pattern detection unit 504, and the dipole estimation unit 505 may be implemented by a hardware circuit (integrated circuit) such as a field-programmable gate array (FPGA) or an application specific integrated circuit (ASIC) instead of a software program.

Note that individual functional units of the information processing device 50 illustrated in FIG. 4 are conceptual illustrations of functions, and are not limited to such configurations. For example, a plurality of functional units illustrated as independent functional units in the information processing device 50 illustrated in FIG. 4 may be configured as one functional unit. On the other hand, the functions of one functional unit in the information processing device 50 illustrated in FIG. 4 may be divided into a plurality of units and configured as a plurality of functional units.

### Flow of Training Processing of Information Processing Device

FIG. 7 is a flowchart illustrating an example of a flow of training processing in the information processing device according to the first embodiment. A flow of training processing of the information processing device 50 according to the present embodiment will be described with reference to FIG. 7.

### Step S11

The acquisition unit 501 acquires waveform data (in this case, waveform data of a MEG signal) to be teaching data for model training in the region detection unit 5041 of the dipole pattern detection unit 504. Subsequently, the processing proceeds to Step S12.

### Step S12

The waveform data is waveform data that has undergone dipole analysis in clinical epilepsy by an expert such as a medical doctor, and thus includes additional information such a time point when characteristic waveform information of IED manifests and sensor selection information. When the sensor selection information does not exist, the sensor selection region may be restored from the selected sensor as described above. Incidentally, the waveform data is not limited to those in clinical use, and may be newly prepared for learning use or may be supplementarily collected using AI. Subsequently, the processing proceeds to Step S13.

### Step S13

Subsequently, the region detection unit 5041 registers, as training data (teaching data), a magnetic isofield map of the waveform data to which a sensor selection region is added as a label. Note that the magnetic isofield map of the waveform data is supposed to have been calculated by the function of the calculation unit 503. Subsequently, the processing proceeds to Step S14.

### Step S14

Subsequently, the region detection unit 5041 performs training using the registered training data (teaching data). Subsequently, the processing proceeds to Step S15.

### Step S15

As a result, the region detection unit 5041 inputs the magnetic isofield map and generates a trained model that outputs a sensor selection region.

Flow of dipole estimation processing in information processing device

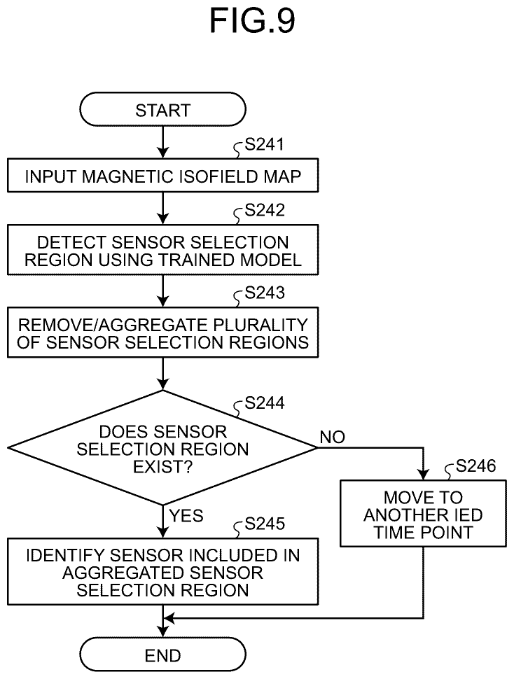

FIG. 8 is a flowchart illustrating an example of a flow of dipole estimation processing of the information processing device according to the first embodiment. FIG. 9 is a flowchart illustrating an example of a flow of dipole pattern detection processing in the information processing device according to the first embodiment. A flow of dipole estimation processing of the information processing device 50 according to the present embodiment will be described with reference to FIGS. 8 and 9.

### Step S21

The acquisition unit 501 acquires waveform data of the MEG signal from the server 40 via the network I/F 105. Subsequently, the processing proceeds to Step S22.

### Step S22

The IED detection unit 502 detects a time point when characteristic waveform information of IED referred to as a spike manifests in the waveform data acquired by the acquisition unit 501. Subsequently, the processing proceeds to Step S23.

### Step S23

Based on the waveform data at the time point detected by the IED detection unit 502, the calculation unit 503 calculates a magnetic isofield map in which regions of equal magnetic field values are expressed by a same luminance value. Subsequently, the processing proceeds to Step S24.

### Step S24

The dipole pattern detection unit 504 detects a dipole pattern region from the magnetic isofield map calculated by the calculation unit 503. That is, the dipole pattern detection unit 504 obtains a sensor selection region surrounding the sensor constituting the dipole pattern region from the magnetic isofield map calculated by the calculation unit 503, thereby identifying the sensor constituting the dipole pattern region. Specifically, processing in the following Steps S241 to S246 is performed.

### Step S241

The region detection unit 5041 of the dipole pattern detection unit 504 performs input of the magnetic isofield map calculated by the calculation unit 503. Subsequently, the processing proceeds to Step S242.

### Step S242

The region detection unit 5041 gives the magnetic isofield map as an input to the trained model, and detects a sensor selection region as an output from the trained model. Subsequently, the processing proceeds to Step S243.

### Step S243

When a plurality of sensor selection regions has been detected by the region detection unit 5041, the aggregating unit 5042 of the dipole pattern detection unit 504 performs removal/aggregating of the plurality of sensor selection regions. Subsequently, the processing proceeds to Step S244.

### Step S244

The determining unit 5043 of the dipole pattern detection unit 504 determines whether the sensor selection region remains as a result of removal and aggregating of the sensor selection region by the aggregating unit 5042. When the sensor selection region exists (Step S244: Yes), the processing proceeds to Step S245. When the sensor selection region does not exist (Step S244: No), the processing proceeds to Step S246.

### Step S245

When the determining unit 5043 has determined that the sensor selection region remains, the sensor identifying unit 5044 of the dipole pattern detection unit 504 identifies a sensor included in the sensor selection region as a sensor constituting the dipole pattern region. Subsequently, Step S24 ends, and the processing proceeds to Step S25.

### Step S246

Due to distortion of the dipole pattern or the like, all the sensor selection regions might be removed as a result of removal/aggregating of the sensor selection regions by the aggregating unit 5042. In this case, it is highly possible that noise is included in the waveform data as a basis of the magnetic isofield map calculated by the calculation unit 503, and accordingly, the processing moves to processing of the IED waveform data at another time point without executing dipole estimation.

### Step S25

The dipole estimation unit 505 performs dipole estimation using a time point when characteristic waveform information of IED manifests, identified by the IED detection unit 502 and waveform data corresponding to the sensor identified by the dipole pattern detection unit 504. This completes the dipole estimation processing.

As described above, the information processing device 50 according to the present embodiment has a configuration in which the acquisition unit 501 acquires waveform data of the MEG signal measured by the plurality of sensors, the calculation unit 503 calculates the magnetic isofield map indicating the distribution of the values of the MEG signal based on the waveform data of a time point when characteristic waveform information of IED manifests among the waveform data acquired by the acquisition unit 501, and the dipole pattern detection unit 504 gives, as an input, the magnetic isofield map calculated by the calculation unit 503 to a model trained using, as the teaching data, data obtained by adding information regarding the sensor selected in the analysis to the magnetic isofield map indicating the distribution of the MEG signal values, obtains a sensor selection region indicating the dipole pattern region as an output, and identifies the sensor constituting the dipole pattern region based on the obtained sensor selection region. In this manner, by training the model using the teaching data being the magnetic isofield map with labeled information of the sensor selected in the analysis by the expert such as a medical doctor, it is possible to identify a sensor close to the sensor selected by the expert. Accordingly, it is possible to more accurately identify the sensor where characteristic waveform information of IED manifests among the plurality of sensors used in the waveform data measurement.

Although the above has described the operation of outputting the sensor selection region by the trained model using the magnetic isofield map based on the waveform data of the MEG signal, the operation is not limited thereto. For example, in the case of waveform data of an EEG signal, it is also possible to train a model by using a current distribution diagram in which regions of equal current values are expressed by a same luminance value, give a current distribution map (an example of distribution information) of waveform data of an EEG signal to the trained model as an input, and obtain a sensor selection region as an output. In this case, the electrode for measuring the EEG signal corresponds to the above-described “sensor”.

### Second Embodiment

A biosignal measurement system 1 according to a second embodiment will be described focusing on differences from the biosignal measurement system 1 according to the first embodiment. The first embodiment above has described the operation using the time point when characteristic waveform information of IED manifests, detected by the IED detection unit 502. Time point detection performed by the IED detection unit 502 is performed, as described above, by using the machine learning model described in Japanese Unexamined Patent Application Publication No. 2021-069929. The time point detected by using such a model is often an inferred time point near a peak zone of the waveform characteristic of IED. In addition, the method described in Japanese Unexamined Patent Application Publication No. 2023-020273 has disclosed a method of searching for a peak zone of a waveform characteristic of the IED. However, depending on the epilepsy lesion, the position of the dipole does not vary between the time point (time point of the peak zone or the like) detected by the IED detection unit 502 and the time point when IED occurs from the actual epileptic lesion in some cases, but can greatly vary in other cases, as described above. In this case, a portion where IED actually occurs in the waveform data is referred to as an onset, and the time point of the portion is referred to as an onset time point. That is, the dipole pattern at the time point of the peak zone may be different from the dipole pattern at the onset time point. Therefore, even when a stable dipole is obtained by the waveform data at the time point of the peak zone with satisfactory S/N, there is a case where the epileptic seizure does not disappear even with excision of the brain region. In consideration of this, the information desired to be obtained by an expert such as a medical doctor is information indicating from which brain region the waveform characteristic of IED has occurred, and it is considered important, for diagnosis of epilepsy, to perform dipole estimation at the onset of waveform data. The present embodiment will describe an operation of detecting the onset time point by searching for the onset based on the time point detected by the IED detection unit 502 and performing dipole estimation using waveform data of the onset time point. The overall configuration of the biosignal measurement system 1 according to the present embodiment and the hardware configuration of the information processing device are similar to the configuration described in the first embodiment. Configuration and operation of functional block of information processing device

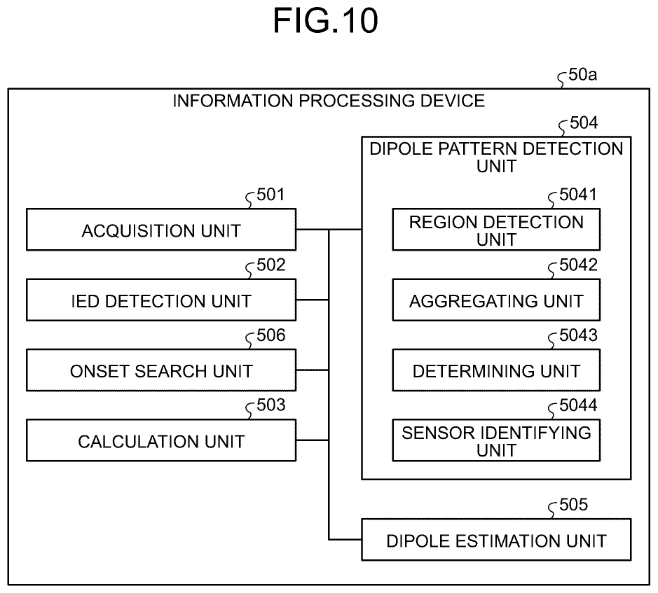

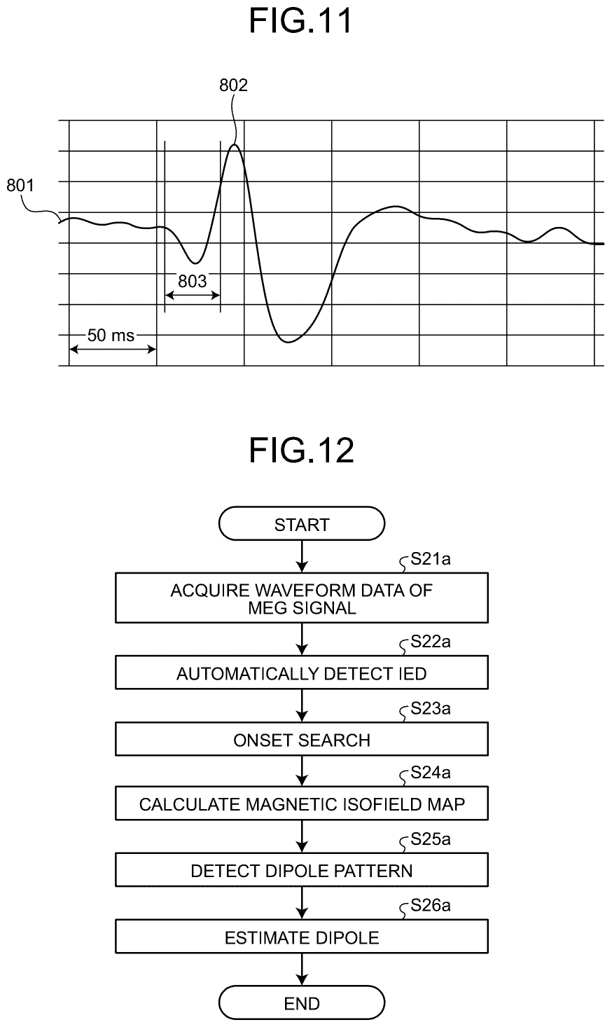

FIG. 10 is a diagram illustrating an example of a configuration of functional blocks of a server according to the second embodiment. FIG. 11 is a diagram illustrating an example of an onset search range in waveform data. The configuration and operation of the functional blocks of an information processing device 50a according to the present embodiment will be described with reference to FIGS. 10 and 11.

As illustrated in FIG. 10, the information processing device 50a includes an acquisition unit 501, an IED detection unit 502, an onset search unit 506, a calculation unit 503, a dipole pattern detection unit 504, and a dipole estimation unit 505. That is, the information processing device 50a further includes the onset search unit 506 in addition to the functional units included in the information processing device 50 according to the first embodiment described above.

The onset search unit 506 is a functional unit that performs dipole estimation for at least one portion corresponding to waveform data before and after the time point detected by the IED detection unit 502, calculates index values such as Goodness of Fit (GoF) of an estimation solution, a Confidence Volume (CV), an amplitude of the waveform, and a dipole distance, and searches for an onset based on the calculated index values. Specifically, the onset search unit 506 detects (searches for) the onset time point based on the index value. At this time, the onset search unit 506 can also normalize the sensor selection region for each time point by using the sensor selection region at at least one of time points before or after the time point detected by the IED detection unit 502. This makes it possible to achieve an improved robustness against sudden noise or artifact.

Here, an operation of the onset search unit 506 will be described using a waveform 801, as a specific waveform, with reference to FIG. 11. The waveform 801 is a waveform obtained when a certain sensor has observed characteristic waveform information of IED. A peak of a waveform including characteristic waveform information in the waveform 801 is illustrated as a peak zone 802. As described above, the time point detected by the IED detection unit 502, that is, the time point detected by the methods described in Japanese Unexamined Patent Application Publication No. 2021-069929 and Japanese Unexamined Patent Application Publication No. 2023-020273, is detected as a time point near the peak zone 802. However, since it is known that the onset which is the portion where the IED actually occurs in the waveform 801 is the time point before the time point of the peak zone 802, the onset search unit 506 searches for the onset in a search range 803 which is a predetermined section earlier than the time point of the peak zone 802. In this case, the onset search unit 506 performs dipole estimation on waveform data at all time points included in the search range 803, calculates the above-described index value, and detects the onset time point as an overall determination.

Based on the waveform data at the onset time point detected by the onset search unit 506, the calculation unit 503 calculates a magnetic isofield map in which regions of equal magnetic field values are represented by a same luminance value.

Note that processing of other functional units of the information processing device 50a is similar to the processing of the information processing device 50 according to the first embodiment described above. Flow of dipole estimation processing in information processing device

FIG. 12 is a flowchart illustrating an example of a flow of dipole estimation processing in the information processing device according to the second embodiment. A flow of dipole estimation processing of the information processing device 50a according to the present embodiment will be described with reference to FIG. 12.

### Steps S21a and S22a

The processing of Steps S21a and S22a is similar to the processing of Steps S21 and S22 (FIG. 8) described above, respectively. Subsequently, the processing proceeds to Step S23a.

### Step S23a

The onset search unit 506 performs dipole estimation for at least one portion corresponding to waveform data (corresponding to a predetermined search range before the time point in a case where the time point is set as the time point of the peak zone) before and after the time point detected by the IED detection unit 502, calculates an index value such as GoF of an estimation solution, the Confidence Volume (CV), an amplitude of a waveform, and a dipole distance, and searches for an onset based on the calculated index value. Specifically, the onset search unit 506 detects the onset time point based on the index value. Subsequently, the processing proceeds to Step S24a.

### Step S24a

Based on the waveform data at the onset time point detected by the onset search unit 506, the calculation unit 503 calculates a magnetic isofield map in which regions of equal magnetic field values are represented by a same luminance value. Subsequently, the processing proceeds to Step S25a.

### Steps S25a and S26a

The processing of Steps S25a and S26a is similar to the processing of Steps S24 and S25 (FIG. 8) described above, respectively. This completes the dipole estimation processing.

Not limited to estimation of a single dipole, evaluating the spread of a plurality of dipoles makes it possible to evaluate information such as whether excision is possible or which region of the brain is to be excised. As described above, in a case where the dipole pattern shifts between the onset and the peak zone, there is a possibility that a dipole cluster cannot be appropriately evaluated in the processing using the time point automatically detected by the IED detection unit 502. However, the evaluation is improved by detecting the onset time point by the onset search unit 506 as described in the present embodiment.

Incidentally, in order to detect the onset time point, the onset search unit 506 performs dipole estimation at each time point of a search period including the onset time point. Therefore, the dipole estimation unit 505 may set a result of dipole estimation already obtained at the onset time point detected by the onset search unit 506 as a final dipole estimation result.

### Third Embodiment

A biosignal measurement system 1 according to a third embodiment will be described focusing on differences from the biosignal measurement system 1 according to the first embodiment. Some cases of epilepsy may include a plurality of epileptic lesions. In this case, making an assumption that there is one current source in dipole estimation would be result in a situation in which automatic detection cannot separate the current sources, leading to a failure in appropriate estimation of the dipole. Accordingly, when a plurality of epileptic lesions is suspected, it is necessary to perform dipole estimation after performing sensor selection separately for the plurality of epileptic lesions. In view of this, the present embodiment will describe an operation, specifically, an operation of identifying a sensor in each sensor selection region in a case where it is determined that a plurality of sensor selection regions exists as a result of processing of removing/aggregating a plurality of sensor selection regions by the aggregating unit 5042 and it is determined that a plurality of dipole patterns exists. The overall configuration of the biosignal measurement system 1 according to the present embodiment, and the hardware configuration and functional block configuration of the information processing device 50 are similar to those described in the first embodiment.

### Flow of Dipole Estimation Processing in Information Processing Device

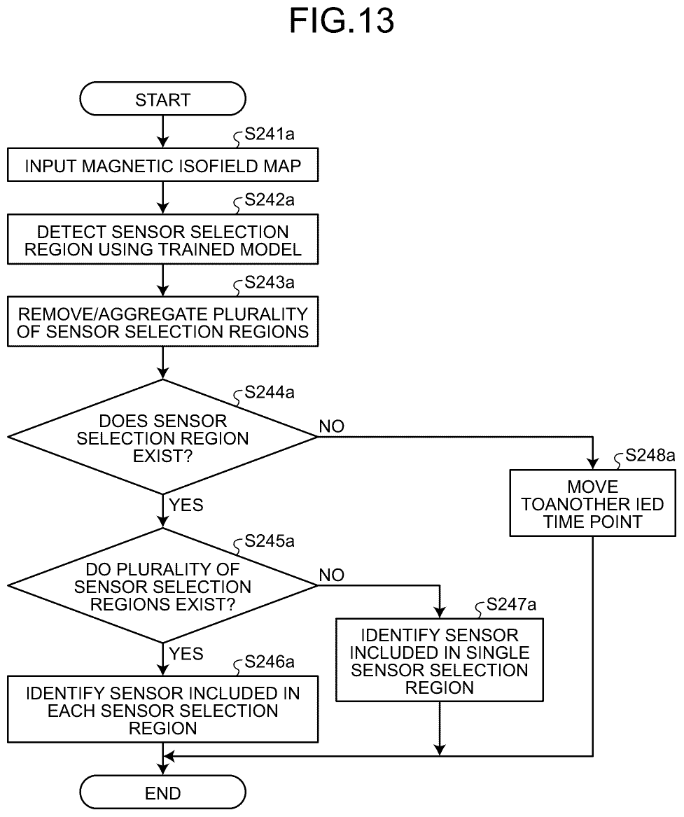

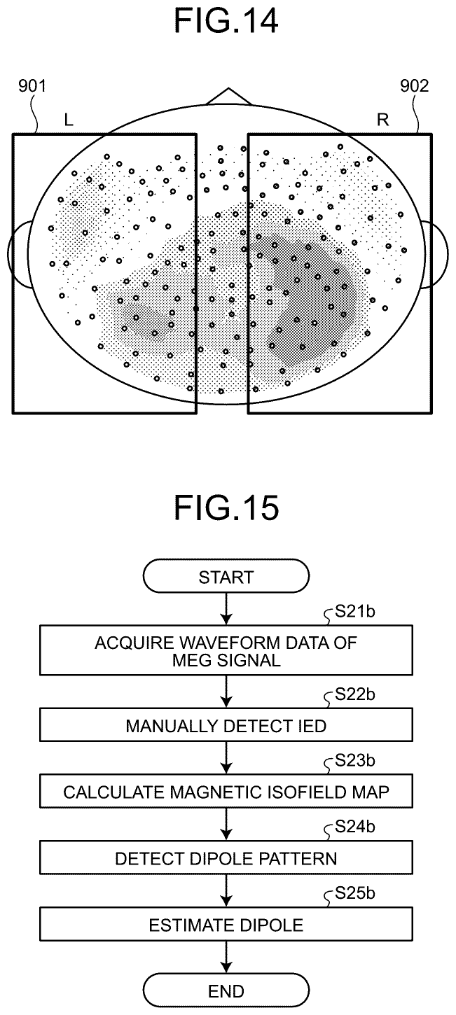

FIG. 13 is a flowchart illustrating an example of a flow of dipole pattern detection processing in an information processing device according to the third embodiment. FIG. 14 is a diagram illustrating an example of a plurality of sensor selection regions corresponding to a dipole pattern. A flow of dipole estimation processing of the information processing device 50 according to the present embodiment will be described with reference to FIGS. 13 and 14. In particular, FIG. 13 illustrates a flow of dipole pattern detection processing by the dipole pattern detection unit 504 of the information processing device 50. The dipole pattern detection unit 504 of the information processing device 50 according to the present embodiment executes detection processing S241a to S248a of a dipole pattern region illustrated in FIG. 13 instead of Step S24 in FIG. 8, specifically, detection processing S241 to S246 of the dipole pattern region illustrated in FIG. 9.

### Steps S241a to S243a

The processing of Steps S241a to S243a is similar to the processing of Steps S241 to S243 (FIG. 9) described above, respectively. Subsequently, the processing proceeds to Step S244a.

### Step S244a

The determining unit 5043 of the dipole pattern detection unit 504 determines whether the sensor selection region remains as a result of removal and aggregating of the sensor selection region by the aggregating unit 5042. When the sensor selection region exists (Step S244 a: Yes), the processing proceeds to Step S245a, and when the sensor selection region does not exist (Step S244a: No), the processing proceeds to Step S248a.

### Step S245a

The determining unit 5043 determines whether a plurality of remaining sensor selection regions exists as a result of removal/aggregating by the aggregating unit 5042. In a case where a plurality of sensor selection regions exists (Step S245a: Yes), the processing proceeds to Step S246a, and in a case where there is a single sensor selection region (Step S245a: No), the processing proceeds to Step S247a.

### Step S246a

When the determining unit 5043 has determined that a plurality of the sensor selection regions exists, the sensor identifying unit 5044 of the dipole pattern detection unit 504 identifies each sensor included in each sensor selection region as a sensor constituting each dipole pattern region. Here, FIG. 14 illustrates an example of a case where the determining unit 5043 determines that two sensor selection regions 901 and 902 exist. In this case, the sensor identifying unit 5044 identifies each of the sensors included in the sensor selection regions 901 and 902 as a sensor constituting each dipole pattern region. Subsequently, Step S24 ends, and the processing proceeds to Step S25 (FIG. 8) described above.

### Step S247a

When the determining unit 5043 determines that a single sensor selection region exists, the sensor identifying unit 5044 identifies a sensor included in the sensor selection region as a sensor constituting the dipole pattern region. Subsequently, Step S24 ends, and the processing proceeds to Step S25.

### Step S248a

The processing of Step S248a is similar to the processing of Step S246 (FIG. 9) described above.

As described above, in the information processing device 50 according to the present embodiment, in a case where a sensor selection region remains as a result of the processing of the aggregating unit 5042, the determining unit 5043 determines whether a plurality of the remaining sensor selection regions exists. In a case where the determining unit 5043 determines that a plurality of sensor selection regions exists, the sensor identifying unit 5044 identifies each sensor included in each sensor selection region as a sensor constituting each dipole pattern region. With this configuration, when a plurality of epileptic lesions is suspected, it is possible to perform sensor selection separately for the plurality of epileptic lesions, thereby performing dipole estimation.

Incidentally, the operation of the present embodiment, that is, the operation of identifying a sensor in each sensor selection region in a case where it is determined that a plurality of sensor selection regions exists as a result of the processing of removing/aggregating the plurality of sensor selection regions by the aggregating unit 5042 and it is determined that a plurality of dipole patterns exists, is also applicable to the above-described second embodiment.

### Fourth Embodiment

A biosignal measurement system 1 according to a fourth embodiment will be described focusing on differences from the biosignal measurement system 1 according to the first embodiment. The first embodiment above has described the operation of automatically detecting the time point when characteristic waveform information of IED manifests, by the IED detection unit 502. In actual analysis, there is a case where a waveform characteristic of IED is visually detected from waveform data of a magnetoencephalograph or the like, or a case where a waveform characteristic of IED is retrieved using an electroencephalograph simultaneously used in the measurement with the magnetoencephalograph or the like as a guide. That is, in a case where the time point at which dipole estimation is to be performed by visual observation is predetermined, it is only necessary to perform sensor selection. Therefore, the present embodiment will describe an operation of manually detecting the time point when characteristic waveform information of IED manifests and appropriately identifying the sensor based on the detected time point. The overall configuration of the biosignal measurement system 1 according to the present embodiment, and the hardware configuration and functional block configuration of the information processing device 50 are similar to those described in the first embodiment.

### Configuration and Operation of Functional Block of Information Processing Device

In the information processing device 50 according to the present embodiment, the IED detection unit 502 detects a time point manually designated by an expert such as a medical doctor as a time point when characteristic waveform information of IED manifests. For example, after performing a designation operation via the input device 106 by an expert who has confirmed the waveform data corresponding to each sensor displayed on the display device 107, that is, an operation of designating the time point determined, by visual observation, as when characteristic waveform information of IED manifests, the IED detection unit 502 detects the time point designated by the designation operation as the time point when characteristic waveform information of IED manifests.

Note that processing of other functional units of the information processing device 50 according to the present embodiment is similar to that of the information processing device 50 according to the first embodiment described above.

### Flow of Dipole Estimation Processing in Information Processing Device

FIG. 15 is a flowchart illustrating an example of a flow of dipole estimation processing of an information processing device according to a fourth embodiment. A flow of dipole estimation processing of the information processing device 50 according to the present embodiment will be described with reference to FIG. 15.

### Step S21b

The processing of Step S21b is similar to the processing of Step S21 (FIG. 8) described above. Subsequently, the processing proceeds to Step S22b. Step S22b

The IED detection unit 502 detects a time point manually designated by an expert such as a medical doctor as a time point when characteristic waveform information of IED manifests. Subsequently, the processing proceeds to Step S23b.

### Steps S23b to 25b

The processing of Steps S23b to 25b is similar to the processing of Steps S23 to S25 (FIG. 8) described above, respectively. This completes the dipole estimation processing.

As described above, in the information processing device 50 according to the present embodiment, the IED detection unit 502 detects the time point manually designated by the expert as the time point when characteristic waveform information of IED manifests. With this configuration, even in a case where the time point of execution of dipole estimation by visual observation is predetermined and only sensor selection is necessary, it is possible to appropriately perform sensor selection with high accuracy.

Note that the operation of detecting the time point manually designated by the IED detection unit 502 in the present embodiment as the time point when characteristic waveform information of IED manifests is also applicable to the second embodiment and the third embodiment described above.

Furthermore, in each of the above-described embodiments, in a case where at least one of the functional units of the information processing devices 50 and 50a is implemented by executing a program, the program is provided by being preinstalled in a ROM chip or the like. Furthermore, in each of the above-described embodiments, the program executed by the information processing devices 50 and 50a may be provided by being recorded, as a file in an installable format or an executable format, in a computer-readable recording medium such as compact disc read only memory (CD-ROM), a flexible disk (FD), a compact disk-recordable (CD-R), or a digital versatile disc (DVD). Furthermore, the configuration may be such that the program to be executed in the information processing devices 50 and 50a in the above-described embodiment is stored in a computer connected to a network such as the Internet, and is provided by being downloaded via the network. Furthermore, the program executed by the information processing devices 50 and 50a in each of the above-described embodiments may be provided or distributed via a network such as the Internet. Furthermore, the program executed by the information processing devices 50 and 50a in each of the above-described embodiments has a module configuration including at least one of the above-described functional units. As an actual hardware configuration, the CPU 101 reads and executes the program from the above-described storage device (for example, the ROM 103, the auxiliary storage device 104, or the like), whereby the above-described functional units are loaded and generated on the main storage device (RAM 102).

The present invention is provided to include the following aspects.
<1>A waveform generation identifying method including:
   acquiring waveform data of a biosignal measured by a plurality of sensors;
   calculating distribution information indicating a distribution of values of the biosignal based on waveform data at a time point when characteristic waveform information of Interictal Epileptiform Discharge (IED) manifests, among the acquired waveform data; and
   giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.
<2> The waveform generation identifying method according to <1>, wherein
   the identifying includes:
      giving the calculated distribution information, as the input, to the model to obtain, as the output, the selection region;
      performing at least one of removal and aggregating of a plurality of selection regions in a case where the selection region includes a plurality of selection regions that have been obtained as the output from the model;
      determining whether a selection region remains as a result of processing in the performing the at least one of removal and aggregating; and
      identifying a sensor included in the selection region as the sensor constituting the dipole pattern region, in a case where the selection region has been determined to remain.
<3> The waveform generation identifying method according to <1> or <2>, further including
   automatically detecting the time point when the characteristic waveform information manifests, from the acquired waveform data,
   wherein at the calculating, the distribution information is calculated based on waveform data at the time point detected at the automatically detecting, among the acquired waveform data.
<4> The waveform generation identifying method according to <1> or <2>, further including
   detecting a time point designated by an operation on an input unit from the acquired waveform data, as the time point when the characteristic waveform information manifests,
   wherein at the calculating, the distribution information is calculated based on waveform data at the time point detected at the detecting, among the acquired waveform data.
<5> The waveform generation identifying method according to <3>, further including
   calculating a predetermined index value related to at least one of portions of the waveform data before and after the time point detected at the detecting, and searching for an onset time point at which the IED has occurred in the waveform data, based on the index value,
   wherein at the calculating, the distribution information is calculated based on waveform data at the onset time point retrieved at the searching, among the acquired waveform data.
<6> The waveform generation identifying method according to <2>, wherein
   at the determining, in a case where the selection region remains as a result of processing of the at least one of removal and aggregating, whether the remaining selection region includes a plurality of remaining selection regions is determined, and
   at the identifying the sensor, in a case where, at the determining, the selection region is determined to include the plurality of selection regions, a sensor included in each of the plurality of selection regions is identified as the sensor constituting one dipole pattern region.
<7> The waveform generation identifying method according to <1> or <2>, wherein
   at the acquiring, the waveform data of a magnetoencephalography (MEG) signal as the biosignal is acquired, and
   at the calculating, a magnetic isofield map is calculated as the distribution information, based on waveform data at the time point when the characteristic waveform information manifests, among the acquired waveform data.
<8> The waveform generation identifying method according to <1> or <2>, wherein
   at the acquiring, the waveform data of an electroencephalography (EEG) signal as the biosignal is acquired, and
   at the calculating, a current distribution map is calculated as the distribution information, based on the waveform data at the time point when the characteristic waveform information manifests, among the acquired waveform data.
<9> The waveform generation identifying method according to <1> or <2>, further including performing dipole estimation using the time point when the characteristic waveform information manifests and the waveform data corresponding to the identified sensor.
<10>A program causing a computer to execute:
   acquiring waveform data of a biosignal measured by a plurality of sensors;
   calculating distribution information indicating a distribution of values of the biosignal based on waveform data at a time point when characteristic waveform information of Interictal Epileptiform Discharge (IED) manifests, among the acquired waveform data; and
   giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.

According to an embodiment, it is possible to accurately identify a sensor where characteristic waveform information of IED manifests among a plurality of sensors used for waveform data measurement.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A waveform generation identifying method comprising:
acquiring waveform data of a biosignal measured by a plurality of sensors;
calculating distribution information indicating a distribution of values of the biosignal based on waveform data at a time point when characteristic waveform information of Interictal Epileptiform Discharge (IED) manifests, among the acquired waveform data; and
giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.

2. The waveform generation identifying method according to claim 1, wherein
the identifying includes:
giving the calculated distribution information, as the input, to the model to obtain, as the output, the selection region;
performing at least one of removal and aggregating of a plurality of selection regions in a case where the selection region includes a plurality of selection regions that have been obtained as the output from the model;
determining whether a selection region remains as a result of processing in the performing the at least one of removal and aggregating; and
identifying a sensor included in the selection region as the sensor constituting the dipole pattern region, in a case where the selection region has been determined to remain.

3. The waveform generation identifying method according to claim 1, further comprising
automatically detecting the time point when the characteristic waveform information manifests, from the acquired waveform data,
wherein at the calculating, the distribution information is calculated based on waveform data at the time point detected at the automatically detecting, among the acquired waveform data.

4. The waveform generation identifying method according to claim 1, further comprising detecting a time point designated by an operation on an input unit from the acquired waveform data, as the time point when the characteristic waveform information manifests,
wherein at the calculating, the distribution information is calculated based on waveform data at the time point detected at the detecting, among the acquired waveform data.

5. The waveform generation identifying method according to claim 3, further comprising
calculating a predetermined index value related to at least one of portions of the waveform data before and after the time point detected at the detecting, and searching for an onset time point at which the IED has occurred in the waveform data, based on the index value,
wherein at the calculating, the distribution information is calculated based on waveform data at the onset time point retrieved at the searching, among the acquired waveform data.

6. The waveform generation identifying method according to claim 2, wherein
at the determining, in a case where the selection region remains as a result of processing of the at least one of removal and aggregating, whether the remaining selection region includes a plurality of remaining selection regions is determined, and
at the identifying the sensor, in a case where, at the determining, the selection region is determined to include the plurality of selection regions, a sensor included in each of the plurality of selection regions is identified as the sensor constituting one dipole pattern region.

7. The waveform generation identifying method according to claim 1, wherein
at the acquiring, the waveform data of a magnetoencephalography (MEG) signal as the biosignal is acquired, and
at the calculating, a magnetic isofield map is calculated as the distribution information, based on waveform data at the time point when the characteristic waveform information manifests, among the acquired waveform data.

8. The waveform generation identifying method according to claim 1, wherein
at the acquiring, the waveform data of an electroencephalography (EEG) signal as the biosignal is acquired, and
at the calculating, a current distribution map is calculated as the distribution information, based on the waveform data at the time point when the characteristic waveform information manifests, among the acquired waveform data.

9. The waveform generation identifying method according to claim 1, further comprising performing dipole estimation using the time point when the characteristic waveform information manifests and the waveform data corresponding to the identified sensor.

10. A non-transitory computer-readable medium including programmed instructions that cause a computer to execute:
acquiring waveform data of a biosignal measured by a plurality of sensors;
calculating distribution information indicating a distribution of values of the biosignal based on waveform data at a time point when characteristic waveform information of Interictal Epileptiform Discharge (IED) manifests, among the acquired waveform data; and
giving, as an input, the distribution information calculated at the calculating, to a model which has been trained using, as teaching data, information obtained by adding information regarding a sensor selected in an analysis, to the distribution information indicating the distribution of the values of the biosignal, to obtain, as an output, a selection region indicating a dipole pattern region, and identifying a sensor constituting the dipole pattern region based on the selection region.
